(19) United States
(12) Patent Application Publication
(10) Pub. No.: US 2013/0184537 A1
(43) Pub. Date: Jul. 18, 2013

(54) MEDICAL IMAGE DIAGNOSIS APPARATUS AND COMPUTER-READABLE MEDIUM

(71) Applicants: Kabushiki Kaisha Toshiba, Tokyo (JP); Toshiba Medical Systems Corportion, Otawara-shi (JP)

(72) Inventors: Nobuyuki KONUMA, Utsunomiya-shi (JP); Megumu Fukuda, Nasushiobara-shi (JP)

(73) Assignees: Toshiba Medical Systems Corporation, Otawara-shi (JP); Kabushiki Kaisha Toshiba, Tokyo (JP)

(21) Appl. No.: 13/742,491

(22) Filed: Jan. 16, 2013

(30) Foreign Application Priority Data
Jan. 16, 2012 (JP) 2012-006400

(51) Int. Cl. A61B 5/00

(52) U.S. Cl. CPC A61B 5/0033
USPC 600/300

(57) ABSTRACT

A medical image diagnosis apparatus according to an embodiment includes: an image data generating unit, an abnormal region detecting unit, a warning data generating unit, and a display unit. The image data generating unit generates clinical-diagnosis image data on the basis of taken image data obtained in a main image taking mode. The image data generating unit also generates failure-diagnosis image data on the basis of taken image data obtained in a failure diagnosis mode. The abnormal region detecting unit detects, from the failure-diagnosis image data, an abnormal region caused by a failure in the apparatus, by comparing the failure-diagnosis image data with reference image data acquired in advance. The warning data generating unit generates warning data on the basis of a result of the abnormal region detection. The display unit displays the clinical-diagnosis image data to which the warning data is appended.

### CROSS-REFERENCE TO RELATED APPLICATION

This application is based upon and claims the benefit of priority from Japanese Patent Application No. 2012-006400, filed on Jan. 16, 2012; the entire contents of which are incorporated herein by reference.

### FIELD

Embodiments described herein relate generally to a medical image diagnosis apparatus and a computer-readable medium that are able to provide various types of information effective for continuous use, on the basis of failure information, failure speculation information, and the like of the apparatus.

### BACKGROUND

Medical image diagnoses using X-ray diagnosis apparatuses, X-ray Computed Tomography (CT) apparatuses, and the like have made rapid progress along with the development of computer technology and are indispensable in today's medical treatments. In particular, X-ray image diagnoses related to the circulatory system have made progress along with the development of catheter manipulations and can be applied to not only the cardiovascular system, but also arteries and veins in the entire body. Usually, an X-ray image diagnosis related to the circulatory system is made by viewing image data acquired during an X-ray image taking process performed on a blood vessel region to which a contrast agent has been administered.

For example, an X-ray diagnosis apparatus used for the purpose of making a diagnosis related to the circulatory system includes: an X-ray generating unit and an X-ray detecting unit (which hereinafter will be collectively referred to as an “image taking system”), as well as a holding unit such as a C-arm that holds the image taking system, and a couchtop on which an examined subject (hereinafter, a “patient”) is placed. By moving the couchtop and the image taking system attached to the holding unit in desired directions, it is possible to perform an X-ray image taking process on an image taking region of the patient from an optimal direction.

Various types of medical image diagnosis apparatuses including the above-mentioned X-ray diagnosis apparatus are provided with a function of detecting and/or speculating a failure occurring in the functional units included in the apparatus. On the basis of detected failure information and/or provided failure speculation information, medical examinations can be canceled, the apparatuses can be substituted with replacement apparatuses, and further, the apparatuses having a failure can be repaired by changing the component parts thereof or the like.

### BRIEF DESCRIPTION OF THE DRAWINGS

FIG. 1 is a block diagram of an overall configuration of a medical image diagnosis apparatus according to an embodiment of the present disclosure;

FIG. 2 is a block diagram of a specific configuration of an image taking unit and a moving mechanism included in the medical image diagnosis apparatus according to the present embodiment;

FIG. 3 is a drawing of a specific example of an abnormal image data list generated by an abnormal image list generating unit according to the present embodiment;

FIG. 4 is a drawing of a specific example of clinical-diagnosis display data generated by a display data generating unit according to the present embodiment;

FIGS. 5A to 5C are drawings of specific examples of a failure diagnosis request dialog, an examination continuation judgment request dialog, and an abnormal region display selection dialog generated by a display data generating unit according to the present embodiment;

FIG. 6 is a drawing of a specific example of a re-interpretation request dialog generated by the display data generating unit according to the present embodiment;

FIG. 7 is a flowchart of a procedure for generating/displaying the clinical-diagnosis display data according to the present embodiment;

FIG. 8 is a flowchart of a procedure for generating/displaying the re-interpretation request dialog according to the present embodiment;

FIG. 9 is a block diagram of an overall configuration of a medical image diagnosis apparatus according to a modification example of the present embodiment;

FIG. 10 is a schematic drawing of an abnormal region setting unit according to the modification example of the present embodiment;

FIG. 11 is a drawing for explaining a positional relationship between a patient and abnormal regions set by an abnormal region setting unit according to the modification example of the present embodiment; and

FIG. 12 is a flowchart of a procedure for generating/displaying clinical-diagnosis image data according to the modification example of the present embodiment.

### DETAILED DESCRIPTION

As mentioned above, the conventional failure detection and failure speculation performed in each medical image diagnosis apparatus are based on the assumption that the apparatus is substituted with a replacement apparatus or that the apparatus having a failure is repaired. Repairing the apparatus, however, raises a first problem where the efficiency of medical examinations is significantly degraded because the medical examinations are interrupted for a long period of time.

When the method of substituting the apparatus with a replacement apparatus is used, it is required to keep a plurality of medical image diagnosis apparatuses having mutually the same functions or capabilities at all times. This raises a second problem where it is difficult to install a plurality of medical image diagnosis apparatuses of the same type when, for example, a required budget for introducing and maintaining the apparatuses is not ensured at an appropriate time at medical facilities in developing countries or at private hospitals. Further, although those medical facilities have a need to continue to perform medical examinations using a medical image diagnosis apparatus having a minor failure or using a medical image diagnosis apparatus that is past the warranty period, conventional medical image diagnosis apparatuses have not been able to sufficiently address the needs of the medical facilities.

In view of these problems hitherto, an object of the present disclosure is to provide a medical image diagnosis apparatus and a computer-readable medium that are able to provide various types of information effective for continuing medical examinations and interpreting medical images, on the basis of failure information, failure speculation related information, and the like of the apparatus that have been acquired.

A medical image diagnosis apparatus according to an embodiment includes: an image data generating unit, an abnormal region detecting unit, a warning data generating unit, and a display unit. The image data generating unit generates clinical-diagnosis image data on the basis of taken image data obtained in a main image taking mode. The image data generating unit also generates failure-diagnosis image data on the basis of taken image data obtained in a failure diagnosis mode. The abnormal region detecting unit detects, from the failure-diagnosis image data, an abnormal region caused by a failure in the apparatus, by comparing the failure-diagnosis image data with reference image data acquired in advance. The warning data generating unit generates warning data on the basis of a result of the abnormal region detection. The display unit displays the clinical-diagnosis image data to which the warning data is appended.

In the following sections, exemplary embodiments of the present disclosure will be explained with reference to the accompanying drawings.

A medical image diagnosis apparatus according to an embodiment of the present disclosure judges the degree of seriousness of a failure, on the basis of failure information or failure speculation related information of functional units included in the medical image diagnosis apparatus. If the failure is minor, the medical image diagnosis apparatus detects, from an image, one or more abnormal regions caused by the failure, by comparing failure-diagnosis image data acquired during an X-ray image taking process in a failure diagnosis mode performed on an image taking region in which no patient is present, with reference image data acquired in advance. Subsequently, the medical image diagnosis apparatus appends warning data indicating that the abnormal regions are present to clinical-diagnosis image data acquired during an X-ray image taking process in a main image taking mode performed on an image taking region of a patient, and further appends, as necessary, position information of the abnormal regions thereto, before causing the image data to be displayed on a display unit. Furthermore, the medical image diagnosis apparatus appends position information of one or more abnormal regions to past image data that was acquired in the past by using the medical image diagnosis apparatus, generates a dialog presenting a list of pieces of past image data from which one or more abnormal regions have been detected and a message prompting re-interpretations of the images, and presents the generated dialog to medical workers who are in charge of interpreting the past image data.

In the present embodiment, a medical image diagnosis apparatus will be explained that is configured to generate various types of image data on the basis of taken image data (hereinafter, “projection data”) acquired during an X-ray image taking process performed on a patient; however, the present disclosure is not limited to this example. For example, the present disclosure is applicable to other medical image diagnosis apparatuses configured to generate image data by implementing other image taking methods such as an X-ray CT image taking method, a Magnetic Resonance Imaging (MRI) image taking method, or the like.

A Configuration of an Apparatus

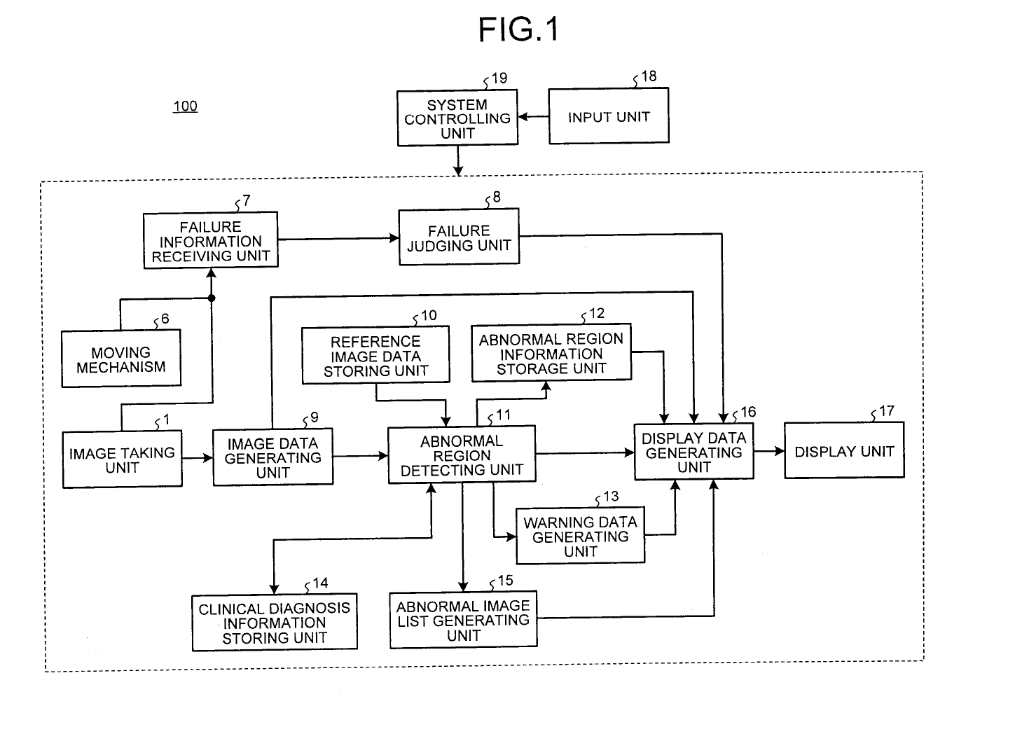

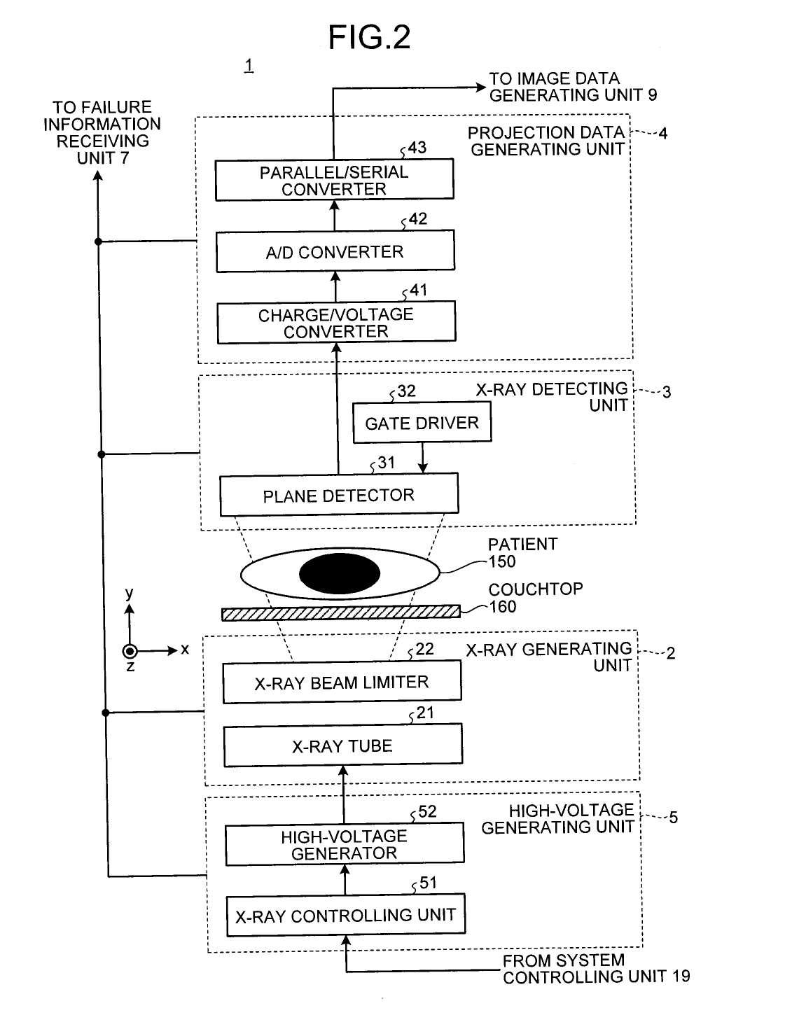

An exemplary configuration and exemplary functions of a medical image diagnosis apparatus according to the present embodiment will be explained, with reference to FIGS. 1 to 6. FIG. 1 is a block diagram of an overall configuration of the medical image diagnosis apparatus according to the present embodiment. FIG. 2 is a block diagram of a specific configuration of an image taking unit and a moving mechanism included in the medical image diagnosis apparatus.

A medical image diagnosis apparatus 100 shown in FIG. 1 includes: an image taking unit 1 configured to generate projection data in a main image taking mode by performing an X-ray image taking process on an image taking region of a patient 150 and to further generate projection data in a failure diagnosis mode by performing a similar X-ray image taking process on an image taking region in which the patient 150 is not present; a holding unit (not shown) configured to hold an X-ray generating unit and an X-ray detecting unit (an image taking system) included in the image taking unit 1; a moving mechanism 6 configured to move the holding unit to which the image taking system is attached and a couchtop on which the patient 150 is placed in predetermined directions; a failure information receiving unit 7 configured to receive failure information supplied from the image taking unit 1 or the moving mechanism 6, as well as information (hereinafter, “failure speculation related information”) about the warranty period, the use starting date, the medical examination dates, and the like of the medical image diagnosis apparatus 100 that is supplied from a system controlling unit 19 (explained later) and is effective for speculating failures; a failure judging unit 8 configured to judge whether or not a failure detected or speculated is a failure that is harmful to the patient 150 or a medical worker (hereinafter, an “operator”) who operates the medical image diagnosis apparatus 100 or a serious failure, on the basis of the failure information and the failure speculation related information; an image data generating unit 9 configured to generate image data (clinical-diagnosis image data and failure-diagnosis image data) by using the projection data supplied from the image taking unit 1 as a result of the X-ray image taking processes in the main image taking mode and the failure diagnosis mode; and a reference image data storing unit 10 configured to store therein reference image data acquired in advance while the patient 150 is not present, by using the medical image diagnosis apparatus 100 at a point in time when no failure had occurred therein.

The medical image diagnosis apparatus 100 further includes: an abnormal region detecting unit 11 configured to detect one or more abnormal regions from the failure-diagnosis image data, by comparing the failure-diagnosis image data generated by the image data generating unit 9 on the basis of the projection data in the failure diagnosis mode acquired during the X-ray image taking process performed on the image taking region in which the patient 150 is not present, with the reference image data read from the reference image data storing unit 10 and to further detect one or more abnormal regions in clinical-diagnosis image data from the past (hereinafter, “past image data”) that was acquired during a past medical examination by using the medical image diagnosis apparatus 100 and is stored in a clinical diagnosis information storing unit 14 (explained later), by comparing the past image data with the failure-diagnosis image data; an abnormal region information storage unit 12 configured to store therein position information and pixel values of the one or more abnormal regions detected from the failure-diagnosis image data; a warning data generating unit 13 configured to, when the abnormal region detecting unit 11 has detected any abnormal region from the failure-diagnosis image data, generate warning data including a predetermined warning message and/or a predetermined warning marker; a clinical diagnosis information storing unit 14 configured to store therein various types of past image data acquired by using the medical image diagnosis apparatus 100; and an abnormal image list generating unit 15 configured to generate a list (“an abnormal image data list”) showing one or more pieces of past image data having one or more abnormal regions, on the basis of a result of the comparison made by the abnormal region detecting unit 11 between pixel values of the past image data and pixel values in the abnormal regions in the failure-diagnosis image data.

The medical image diagnosis apparatus 100 further includes: a display data generating unit 16 configured to generate clinical-diagnosis display data by appending the warning data generated by the warning data generating unit 13 in the failure diagnosis mode and position information of the abnormal regions detected by the abnormal region detecting unit 11 to the clinical-diagnosis image data generated by the image data generating unit 9 in the main image taking mode and to further generate various types of dialogs on the basis of a result of the failure judgment by the failure judging unit 8, a result of the abnormal region detection by the abnormal region detecting unit 11, and the like; a display unit 17 configured to display the clinical-diagnosis display data and the various types of dialogs generated by the display data generating unit 16; an input unit 18 configured to input patient information and medical examination information, to set various types of image taking conditions including an X-ray radiation condition, and to input various types of instruction signals for, for example, instructing a failure diagnosis and instructing continuation/cancellation of medical examinations, as well as instructing that the abnormal regions should be displayed in the clinical-diagnosis image data; and a system controlling unit 19 configured to control the functional units described above in an integrating manner.

The image taking unit 1 includes: the X-ray generating unit and the X-ray detecting unit structuring the image taking system; a projection data generating unit; a high-voltage generating unit; the holding unit that holds the image taking system; and the couchtop on which the patient 150 is placed. The image taking unit 1 has a function of generating the projection data on the basis of the amount of X-rays that have passed through the patient 150.

FIG. 2 is a block diagram of a specific configuration of the above-mentioned functional units included in the image taking unit 1. An X-ray generating unit 2 includes: an X-ray tube 21 configured to radiate X-rays onto the image taking region of the patient 150 in the main image taking mode and onto the image taking region in which the patient 150 is not present in the failure diagnosis mode; and an X-ray beam limiter 22 configured to form X-ray cone beams with respect to the X-rays emitted from the X-ray tube 21. The X-ray tube 21 is a vacuum tube that generates the X-rays and is configured to generate the X-rays by causing thermoelectrons generated from a heated cathode (a filament) to be accelerated by a direct-current high voltage supplied from a high-voltage generating unit 5 and to collide with a tungsten anode. The X-ray beam limiter 22 is used for the purpose of reducing the exposure dose of the patient 150 and improving the image quality of the image data and is configured so as to include the following (not shown): a limiting vane (an upper vane) configured to limit the X-rays emitted from the X-ray tube 21 to a predetermined radiation region; a lower vane configured to, by moving in conjunction with the limiting vane, reduce scattered beams and a leakage radiation dose; and a compensation filter configured to prevent halation by selectively reducing X-rays that have passed through media having a low absorption amount.

The X-ray detecting unit 3 can be configured by using an image intensifier and an X-ray TV or by using a Flat Panel Detector (FPD). Examples of the FPD include a type that directly converts X-rays into electric charges and a type that once converts X-rays into light before converting the light into electric charges. In the present example, the X-ray detecting unit 3 will be explained as including a FPD capable of directly converting X-rays into electric charges; however, the present disclosure is not limited to this example.

In other words, the X-ray detecting unit 3 according to the present embodiment includes, as shown in FIG. 2, a FPD 31 configured to detect X-rays that have passed through the patient 150; and a gate driver 32 configured to supply a drive signal to the FPD 31, the drive signal causing the X-rays detected by the FPD 31 to be read as signal electric charges.

The FPD 31 is configured by two-dimensionally arranging small detecting elements in a column direction and a line direction. Each of the detecting elements includes the following (not shown): a photoelectric film configured to sense X-rays and to generate a signal electric charge in accordance with the amount of incident X-rays; a charge storing capacitor configured to store therein the signal electric charge generated by the photoelectric film; and a Thin Film Transistor (TFT) configured to read the signal electric charge stored in the charge storing capacitor at a predetermined time.

A projection data generating unit 4 includes: a charge/voltage converter 41 configured to convert the signal electric charges that are read from the FPD 31, for example, in parallel in units along the line direction; an Analog/Digital (A/D) converter 42 configured to convert outputs of the charge/voltage converter 41 into digital signals (data elements of the projection data); and a parallel/serial converter 43 configured to convert the above-mentioned data elements resulting from the digital conversion into time-series data elements. Further, the time-series data elements output from the parallel/serial converter 43 are supplied to the image data generating unit 9.

The high-voltage generating unit 5 includes: a high-voltage generator 52 configured to generate the high-voltage to be applied to between the anode and the cathode for the purpose of accelerating the thermoelectrons generated from the cathode of the X-ray tube 21; and an X-ray controlling unit 51 configured to control a tube current, a tube voltage, an application time period, application timing, an application repeating cycle, and the like of the high-voltage generator 52 on the basis of the X-ray radiation condition and an X-ray radiation timing signal supplied from the system controlling unit 19. Under the control of the X-ray controlling unit 51, the X-rays having predetermined radiation energy are emitted from the X-ray tube 21 toward the FPD 31 included in the X-ray detecting unit 3.

Further, the X-ray generating unit 2, the X-ray detecting unit 3, the projection data generating unit 4, and the high-voltage generating unit 5 each have a failure detecting unit (not shown) configured to detect failures in the functional unit. The failure information detected by these failure detecting units is supplied to the failure judging unit 8 via the failure information receiving unit 7. The holding unit configured to hold the X-ray generating unit 2 and the X-ray detecting unit 3 (the image taking system) is omitted from FIG. 2.

Returning to the description of FIG. 1, the moving mechanism 6 includes the following (not shown): a holding unit moving mechanism configured to turn or move, around the patient 150, the holding unit to which the X-ray generating unit 2 and the X-ray detecting unit 3 (the image taking system) are attached; a couchtop moving mechanism configured to move a couchtop 160 on which the patient 150 is placed in a body axis direction (the z-direction in FIG. 2) of the patient 150 and directions orthogonal to the body axis (the x-direction and the y-direction in FIG. 2); and a moving mechanism controlling unit configured to control the holding unit moving mechanism and the couchtop moving mechanism.

The moving mechanism controlling unit is configured to supply, to the holding unit moving mechanism, a moving control signal generated on the basis of an image taking system moving instruction signal supplied from the input unit 18 via the system controlling unit 19 and is configured to set an image taking position and an image taking direction to be used during an X-ray image taking process, by turning or moving, around the patient 150, the holding unit to which the image taking system is attached.

Further, the moving mechanism controlling unit is configured to supply, to the couchtop moving mechanism, a moving control signal generated on the basis of a couchtop moving instruction signal supplied via the system controlling unit 19 and is configured to set the center of an image taking region by parallel-shifting the couchtop 160 in the body axis direction of the patient 150 or in the directions orthogonal to the body axis.

Further, the holding unit moving mechanism, the couchtop moving mechanism, and the moving mechanism controlling unit each have a failure detecting unit configured to detect failures in the functional unit. The failure information detected by these failure detecting units is supplied to the failure judging unit 8 via the failure information receiving unit 7.

After that, the failure information receiving unit 7 shown in FIG. 1 receives the failure information (the result of the failure detection) supplied from any of the failure detecting units included in the image taking unit 1 or the moving mechanism 6, as well as the failure speculation related information (i.e., the information about the warranty period, the use starting date, the medical examination dates, and the like of the medical image diagnosis apparatus 100) supplied from the system controlling unit 19. On the basis of the failure information and the failure speculation related information supplied from the failure information receiving unit 7, the failure judging unit 8 judges whether or not the detected or speculated failure is a serious failure or a harmful failure for the patient 150 or the operator.

For example, if failure information indicating a possibility that the holding unit or the couchtop 160 may operate out of control is supplied from the moving mechanism 6 or if failure information indicating a possibility that a dose of X-rays larger than necessary may be radiated onto the patient 150 is supplied from the high-voltage generating unit 5 or the X-ray generating unit 2, the failure judging unit 8 determines that a failure that can be seriously harmful to the patient 150 is occurring in the functional unit and supplies an instruction signal indicating that no medical examination should be performed by using the medical image diagnosis apparatus 100, to the system controlling unit 19.

As another example, if failure information indicating a possibility that an abnormal region such as a missing region may occur in a part of the image data is supplied from the X-ray detecting unit 3 or the projection data generating unit 4, the failure judging unit 8 determines that the failure occurring in the functional unit is a minor failure that is not harmful to the patient 150 and supplies an instruction signal (a failure diagnosis request instruction signal) to request a failure diagnosis for the purpose of evaluating the image data acquired by using the medical image diagnosis apparatus 100 having such a failure, to a dialog generating unit (explained later) included in the display data generating unit 16.

The image data generating unit 9 includes a projection data storage unit and an image processing unit (not shown). In correspondence with the line direction and the column direction of the detecting elements, the projection data storage unit is configured to sequentially store therein the data elements of the projection data that are output along a time-series from the parallel/serial converter 43 included in the projection data generating unit 4 in the main image taking mode and the failure diagnosis mode, so that two-dimensional projection data is formed. Further, the image processing unit performs, as necessary, a predetermined affine transformation process, a filtering process for the purpose of reducing noise or emphasizing contours, a gamma correction process, and/or the like on the two-dimensional projection data read from the projection data storage unit, so as to generate the clinical-diagnosis image data and the failure-diagnosis image data.

Further, the reference image data storing unit 10 has stored therein, in advance, the reference image data acquired in the past during the X-ray image taking process in the failure diagnosis mode performed on the predetermined image taking region in which the patient 150 is not present, by using the medical image diagnosis apparatus 100 having no failure. The abnormal region detecting unit 11 includes a data comparing unit having a subtraction processing function and a data appending unit (not shown). The data comparing unit performs a subtraction process between the failure-diagnosis image data which the image data generating unit 9 generates on the basis of the projection data acquired during the X-ray image taking process in the failure diagnosis mode and the reference image data read from the reference image data storing unit 10. The data comparing unit thereby detects, for example, one or more abnormal regions caused by a failure in the detecting elements included in the FPD 31 from the failure-diagnosis image data. Further, the position information, the pixel values, and the like of the detected abnormal regions are stored into the abnormal region information storage unit 12. Also, when one or more abnormal regions have been detected from the failure-diagnosis image data, the abnormal region detecting unit 11 supplies an instruction signal (an examination continuation judgment request instruction signal) to request that a judgment should be made as to whether the medical examination should be continued or not, to the dialog generating unit included in the display data generating unit 16.

In the present example, when one or more abnormal regions have been detected, the failure judging unit 8 supplies the examination continuation judgment request instruction signal to the dialog generating unit included in the display data generating unit 16; however, the exemplary embodiments are not limited to this example. For example, it is acceptable to configure the failure judging unit 8 so as to determine whether an examination continuation judgment request instruction signal should be supplied or not, depending on the sizes and/or the positions of the detected abnormal regions. In this situation, the size of each of the abnormal regions is, for example, the number of pixels included in the abnormal region.

In a specific example, for instance, if the number of pixels included in an abnormal region exceeds a predetermined value, the failure judging unit 8 supplies an examination continuation judgment request instruction signal to the dialog generating unit included in the display data generating unit 16. In this situation, for example, when two or more abnormal regions have been detected, the failure judging unit 8 supplies an examination continuation judgment request instruction signal, if a total value of the number of pixels included in the abnormal regions exceeds a predetermined value.

In another example, if the position of an abnormal region is near the center of the image data, the failure judging unit 8 supplies an examination continuation judgment request instruction signal to the dialog generating unit included in the display data generating unit 16. In this situation, for example, the failure judging unit 8 sets a region having a predetermined size near the center of the image data and counts the pixels in the abnormal region included in the set region. Further, if the number of counted pixels exceeds a predetermined value, the failure judging unit 8 supplies an examination continuation judgment request instruction signal.

Further, the data comparing unit included in the abnormal region detecting unit 11 detects whether any abnormal region is present in the past image data or not, by comparing the pixel values of the past image data that was acquired during a past medical examination performed by using the medical image diagnosis apparatus 100 and is stored in the clinical diagnosis information storing unit 14, with the pixel values in the abnormal regions in the failure-diagnosis image data. In other words, if the past image data and the failure-diagnosis image data are acquired by using the FPD 31 having mutually the same defective location, the pixel values in the abnormal regions in the failure-diagnosis image data are substantially equal to the pixel values of the past image data corresponding to the abnormal regions. Thus, by comparing the pixel values in the corresponding sites between the two pieces of image data, it is possible to detect whether any abnormal region is present in the past image data or not and the position information thereof.

Further, if one or more abnormal regions have been detected, the data appending unit appends the position information of the abnormal regions detected by the data comparing unit to the past image data read from the clinical diagnosis information storing unit 14 and stores the past image data back into the clinical diagnosis information storing unit 14. Further, the data appending unit supplies an instruction signal (a re-interpretation request instruction signal) to request a re-interpretation of the past image data from which the abnormal regions were detected, to the dialog generating unit included in the display data generating unit 16.

After that, when one or more abnormal regions have been detected from the failure-diagnosis image data by the abnormal region detecting unit 11, the warning data generating unit 13 generates warning data indicating that one or more abnormal regions that may hinder the interpretation are present in the clinical-diagnosis image data in the main image taking mode acquired by using a functional unit having a failure.

The clinical diagnosis information storing unit 14 stores therein the various types of past image data acquired during past medical examinations performed by the medical image diagnosis apparatus 100, together with additional information, e.g., patient information such as a “patient ID”, and a “patient name”, as well as medical examination information such as an “examination date”, an “examination time”, and an “examined region”.

The abnormal image list generating unit 15 generates a list (an abnormal image data list) showing one or more pieces of past image data having one or more abnormal regions found by the data comparing unit included in the abnormal region detecting unit 11, by comparing the pixel values of the past image data read from the clinical diagnosis information storing unit 14 with the pixel values in the abnormal regions in the failure-diagnosis image data read from the abnormal region information storage unit 12, on the basis of identification information and the additional information thereof.

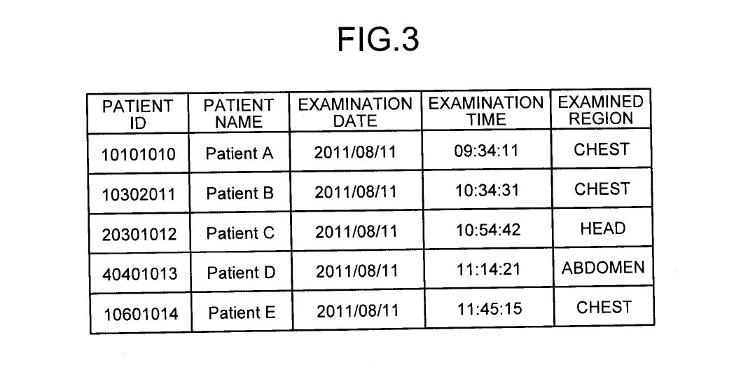

FIG. 3 illustrates a specific example of the abnormal image data list generated by the abnormal image list generating unit 15. The abnormal image data list shows the additional information (e.g., the patient information such as a “patient ID” and a “patient name”, as well as the medical examination information such as an “examination date”, an “examination time”, and an “examined region”) of each of the pieces of past image data that have at least one abnormal region and that were extracted from the past image data stored in the clinical diagnosis information storing unit 14.

Further, the display data generating unit 16 shown in FIG. 1 includes a data combining unit and the dialog generating unit (not shown). The data combining unit is configured to generate the clinical-diagnosis display data by appending the warning data generated by the warning data generating unit 13 to the clinical-diagnosis image data acquired during the X-ray image taking process in the main image taking mode and further appending, as necessary, the position information of the abnormal regions detected by the data comparing unit included in the abnormal region detecting unit 11 by comparing the reference image data with the failure-diagnosis image data.

The dialog generating unit included in the display data generating unit 16 has a function of generating various types of dialogs, on the basis of a result of the failure judgment by the failure judging unit 8, a result of the abnormal region detection by the abnormal region detecting unit 11, and the like and is configured to generate a failure diagnosis request dialog to prompt a failure diagnosis for the purpose of evaluating the abnormal regions, on the basis of the failure diagnosis request instruction signal supplied from the failure judging unit 8. Further, the dialog generating unit is configured to generate an examination continuation judgment request dialog to request that a judgment should be made as to whether the medical examination should be continued or canceled, on the basis of the examination continuation judgment request instruction signal supplied from the abnormal region detecting unit 11.

Further, when the data combining unit generates the clinical-diagnosis display data by appending the warning data generated by the warning data generating unit 13 to the clinical-diagnosis image data in the main image taking mode supplied from the image data generating unit 9, the dialog generating unit generates an abnormal region display selection dialog used for having the user select whether the abnormal regions should be displayed or not with the clinical-diagnosis image data. Also, when one or more abnormal regions caused by a failure have been detected from any of the past image data that has already been interpreted and is stored in the clinical diagnosis information storing unit 14, the dialog generating unit generates a re-interpretation request dialog to request that the past image data from which the one or more abnormal regions were detected should be re-interpreted, on the basis of the re-interpretation request instruction signal supplied from the abnormal region detecting unit 11.

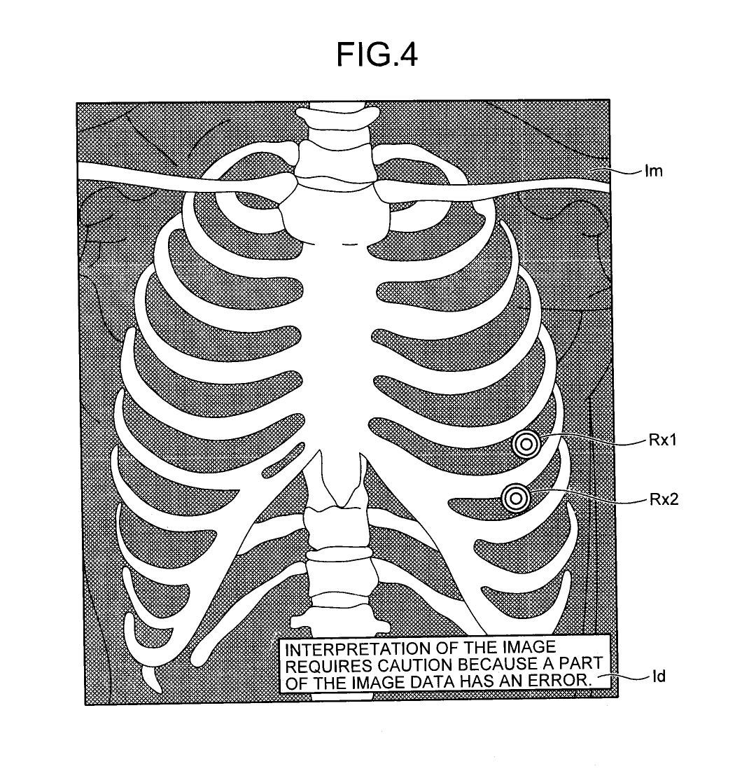

Next, the clinical-diagnosis display data generated by the data combining unit and various types of dialogs generated by the dialog generating unit will be explained, with reference to FIGS. 4 to 6.

FIG. 4 illustrates a specific example of the clinical-diagnosis display data generated by the data combining unit included in the display data generating unit 16. The clinical-diagnosis display data is generated by appending a warning message Id such as “Interpretation of the image requires caution because a part of the image data has an error.” generated as warning data by the warning data generating unit 13, to clinical-diagnosis image data Im in the main image taking mode generated by the image data generating unit 9 and by further appending thereto, as necessary, abnormal region markers Rx1, Rx2, and so on, indicating the position information of the abnormal regions (i.e., on the basis of an abnormal region display instruction signal supplied from the input unit 18 in response to the abnormal region display selection dialog displayed on the display unit 17).

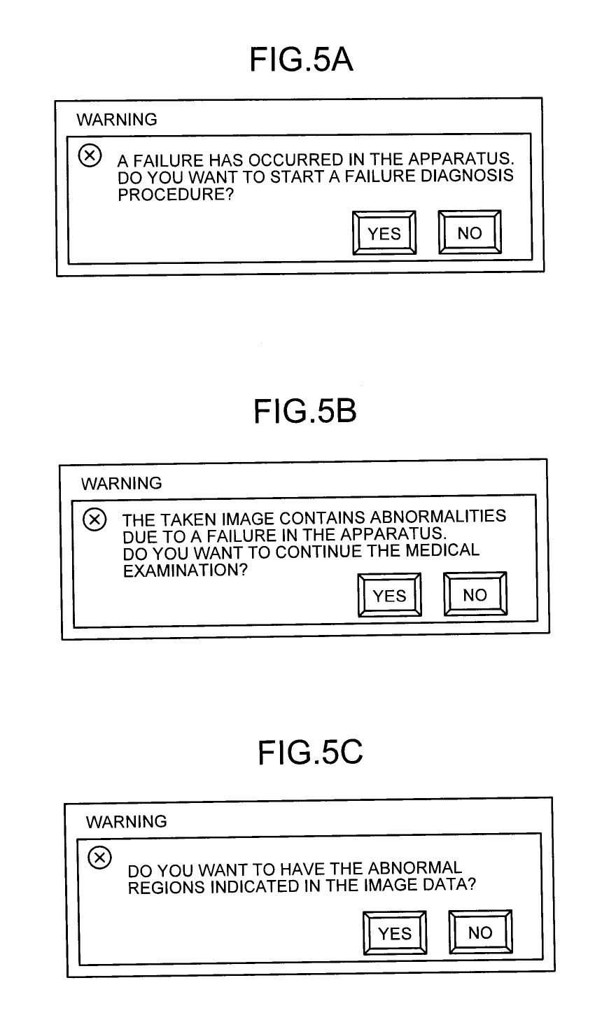

FIG. 5A illustrates a specific example of the failure diagnosis request dialog generated by the dialog generating unit included in the display data generating unit 16, on the basis of the failure diagnosis request instruction signal supplied from the failure judging unit 8, when the failure judging unit 8 that received the failure information and/or the failure speculation related information supplied from the failure information receiving unit 7 has determined that the failure detected or speculated by the image taking unit 1 or the moving mechanism 6 is a minor failure that will not be harmful to the patient 150 or the operator. The failure diagnosis request dialog presents a message such as “A failure has occurred in the apparatus. Do you want to start a failure diagnosis procedure?” to prompt a failure diagnosis for the purpose of evaluating the abnormal regions.

FIG. 5B illustrates a specific example of the examination continuation judgment request dialog generated by the dialog generating unit on the basis of the examination continuation judgment request instruction signal supplied from the abnormal region detecting unit 11, when the abnormal region detecting unit 11 has detected, from the failure-diagnosis image data, one or more abnormal regions caused by a failure in the FPD 31 or the like, by performing the subtraction process between the failure-diagnosis image data and the reference image data. The examination continuation judgment request dialog presents a message such as “The taken image contains abnormalities due to a failure in the apparatus. Do you want to continue the medical examination?” to request that a judgment should be made as to whether the medical examination should be continued or canceled.

Further, FIG. 5C illustrates a specific example of the abnormal region display selection dialog generated by the dialog generating unit, when the data combining unit included in the display data generating unit 16 generates the clinical-diagnosis display data by appending the warning data generated by the warning data generating unit 13 to the clinical-diagnosis image data in the main image taking mode supplied from the image data generating unit 9. The abnormal region display selection dialog presents a message such as “Do you want to have the abnormal regions indicated in the image data?”, to prompt a selection regarding whether the position information of the abnormal regions that were detected from the failure-diagnosis image data and is stored in the abnormal region information storage unit 12 should be further appended to the clinical-diagnosis display data.

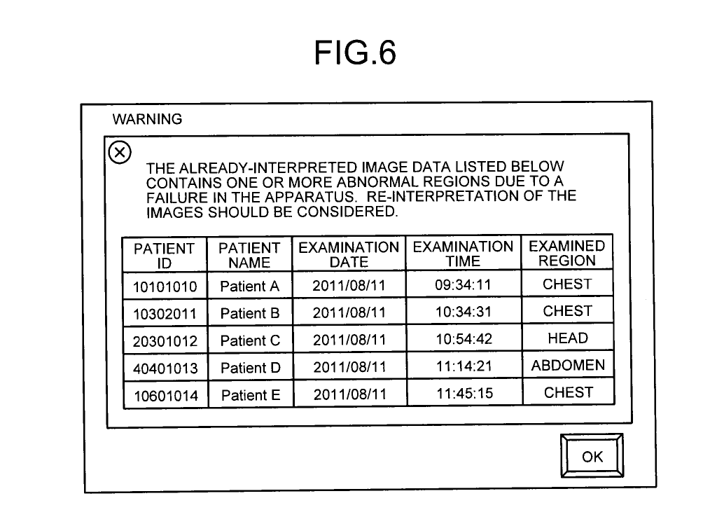

FIG. 6 illustrates a specific example of the re-interpretation request dialog generated by the dialog generating unit on the basis of the re-interpretation request instruction signal supplied from the abnormal region detecting unit 11, when the data comparing unit included in the abnormal region detecting unit 11 has detected one or more abnormal regions from the past image data by comparing the pixel values of the image data read from the clinical diagnosis information storing unit 14 with the pixel values in the abnormal regions in the failure-diagnosis image data read from the abnormal region information storage unit 12. The re-interpretation request dialog presents the abnormal image data list generated by the abnormal image list generating unit 15 and a message such as “The already-interpreted image data listed below contains one or more abnormal regions due to a failure in the apparatus. Re-interpretation of the images should be considered.” to prompt a re-interpretation of the past image data shown in the abnormal image data list.

Further, the display unit 17 shown in FIG. 1 includes a data converting unit and a monitor (not shown). The data converting unit is configured to perform a converting process such as a Digital/Analog (D/A) conversion or a television format conversion on the clinical-diagnosis display data (see FIG. 4) and the various types of dialogs (see FIGS. 5A, 5B, and 6) generated by the display data generating unit 16 and to cause the result of the conversion to be displayed on the monitor.

The input unit 18 is an interactive interface that includes one or more input devices such as a display panel, a keyboard, a trackball, a joystick, a mouse, and the like. The input unit 18 is configured to input the patient information and the medical examination information, to set various types of image taking conditions including the X-ray radiation condition, to set an image data generation condition and a display data generation condition, to instruct a failure diagnosis (to select the failure diagnosis mode), to instruct that a medical examination should be continued or canceled, as well as to instruct that the abnormal regions should be displayed with the clinical-diagnosis image data.

For example, by using the input devices described above, the operator of the medical image diagnosis apparatus 100 selects the failure diagnosis mode in response to the failure diagnosis request dialog shown in FIG. 5A that was generated by the dialog generating unit included in the display data generating unit 16 and is displayed on the display unit 17, and also, instructs that the medical examination should be continued or canceled in response to the examination continuation judgment request dialog shown in FIG. 5B. In another example, when the abnormal region detecting unit 11 has detected one or more abnormal regions from the failure-diagnosis image data, the operator instructs whether or not the position information of the abnormal regions should be appended to the clinical-diagnosis image data acquired in the main image taking mode, in response to the abnormal region display selection dialog shown in FIG. 5C displayed on the display unit 17.

The system controlling unit 19 includes a Central Processing Unit (CPU) and an input information storage unit (not shown). The various types of information described above input/set by the input unit 18 are stored into the input information storage unit. Further, the input information storage unit has stored therein, in advance, the failure speculation related information such as the warranty period, the use starting date, the medical examination dates, and the like of the medical image diagnosis apparatus 100. Further, the CPU controls the functional units described above included in the medical image diagnosis apparatus 100 in an integrating manner, on the basis of the stored information, so that abnormal regions are detected on the basis of the failure-diagnosis image data and so that the abnormal region information is appended to the clinical-diagnosis image data acquired in the main image taking mode and to the past image data stored in the clinical diagnosis information storing unit 14.

A procedure for Generating/Displaying the Clinical-Diagnosis Display Data

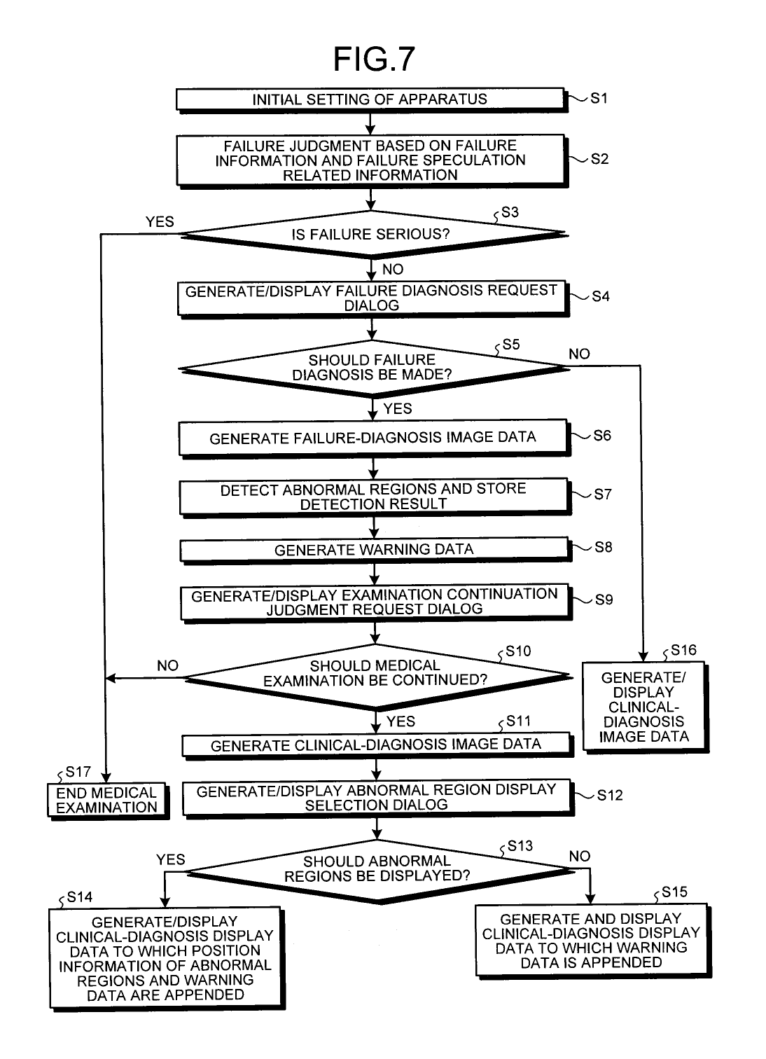

Next, a procedure for generating/displaying the clinical-diagnosis display data according to the present embodiment will be explained, with reference to the flowchart in FIG. 7.

Prior to generation of the clinical-diagnosis display data, the operator of the medical image diagnosis apparatus 100 inputs the patient information and the medical examination information through the input unit 18 and subsequently sets, for example, the various types of image taking conditions including the X-ray radiation condition as well as the image data generation condition and the display data generation condition. The input information and the setting information are stored into an input information storage unit included in the system controlling unit 19 (step S1 in FIG. 7).

The failure judging unit 8 receives failure information (a result of the failure detection) supplied from any of the failure detecting units included in the image taking unit 1 or the moving mechanism 6 via the failure information receiving unit 7 as well as failure speculation related information supplied from the system controlling unit 19 and judges the degree of seriousness of a failure detected or speculated, on the basis of the received information (step S2 in FIG. 7). Further, if the failure judging unit 8 has determined that the failure is a minor failure that is not harmful to the patient 150 or the operator (step S3, NO in FIG. 7), the failure judging unit 8 transmits a failure diagnosis request instruction signal to the dialog generating unit included in the display data generating unit 16. Having received the instruction signal, the dialog generating unit generates a failure diagnosis request dialog (see FIG. 5A) to prompt a failure diagnosis for the purpose of evaluating the abnormal regions occurring in the image data due to the above-mentioned failure and causes the generated dialog to be displayed on the display unit 17 (step S4 in FIG. 7).

Subsequently, when a failure diagnosis execution instruction signal in response to the failure diagnosis request dialog or a failure diagnosis request signal based on the operator's intention is input through the input unit 18 (step S5, YES in FIG. 7), the image taking unit 1 receives the instruction signal or the request signal via the system controlling unit 19 and generates projection data by performing an X-ray image taking process in the failure diagnosis mode on a predetermined image taking region in which the patient 150 is not present. The image data generating unit 9 generates failure-diagnosis image data by performing a predetermined process on the obtained projection data (step S6 in FIG. 7).

After that, the abnormal region detecting unit 11 detects one or more abnormal regions from the failure-diagnosis image data by performing a subtraction process between the failure-diagnosis image data generated by the image data generating unit 9 on the basis of the projection data acquired during the X-ray image taking process in the failure diagnosis mode and the reference image data read from the reference image data storing unit 10. Further, the abnormal region detecting unit 11 stores a detection result (the position information and the pixel values of the abnormal regions) into the abnormal region information storage unit 12 (step S7 in FIG. 7).

When the abnormal region detecting unit 11 has detected one or more abnormal regions from the failure-diagnosis image data, the warning data generating unit 13 generates warning data indicating that the clinical-diagnosis image data in the main image taking mode acquired by using a functional unit having a failure contains one or more abnormal regions that may hinder the interpretation of the image (step S8 in FIG. 7).

In addition, when the one or more abnormal regions have been detected from the failure-diagnosis image data, the abnormal region detecting unit 11 supplies an examination continuation judgment request instruction signal to the dialog generating unit included in the display data generating unit 16. Having received the instruction signal, the dialog generating unit generates an examination continuation judgment request dialog (see FIG. 5B) to request that a judgment should be made as to whether the medical examination should be continued or canceled and causes the generated dialog to be displayed on the display unit 17 (step S9 in FIG. 7).

After that, if an examination continuation instruction signal in response to the examination continuation judgment request dialog is input and the main image taking mode is selected through the input unit 18 (step S10, YES in FIG. 7), the image taking unit 1 receives the instruction signal and the selection signal via the system controlling unit 19 and generates projection data by performing an X-ray image taking process in the main image taking mode on a predetermined image taking region of the patient 150 placed on the couchtop 160. The image data generating unit 9 generates clinical-diagnosis image data by performing a predetermined process on the obtained projection data (step S11 in FIG. 7).

Further, the dialog generating unit included in the display data generating unit 16 generates an abnormal region display selection dialog (see FIG. 5C) to prompt the operator to select whether the abnormal regions should be displayed or not displayed with the clinical-diagnosis image data and causes the generated dialog to be displayed on the display unit 17 (step S12 in FIG. 7). After that, if an abnormal region display instruction signal in response to the abnormal region display selection dialog is input through the input unit 18 (step S13, YES in FIG. 7), the data combining unit included in the display data generating unit 16 receives the instruction signal via the system controlling unit 19, further generates clinical-diagnosis display data (first clinical-diagnosis display data) by appending warning data generated by the warning data generating unit 13 and the position information of the abnormal regions read from the abnormal region information storage unit 12 to the clinical-diagnosis image data acquired during the X-ray image taking process in the main image taking mode, and causes the generated display data to be displayed on the display unit 17 (step S14 in FIG. 7).

In contrast, if an abnormal region non-display instruction signal is input through the input unit 18, the data combining unit receives the instruction signal, further generates clinical-diagnosis display data (second clinical-diagnosis display data) by appending warning data to the clinical-diagnosis image data, and causes the generated display data to be displayed on the display unit 17 (step S15 in FIG. 7).

If no failure diagnosis execution instruction signal is input through the input unit 18 in response to the failure diagnosis request dialog displayed at step S4 above (step S5, NO in FIG. 7), only the clinical-diagnosis image data generated by the image data generating unit 9 is displayed on the display unit 17 (step S16 in FIG. 7). Further, if the failure is determined to be a serious failure as a result of the failure judgment at step S2 (step S3, YES in FIG. 7), or if no examination continuation instruction signal is input in response to the examination continuation judgment request dialog displayed at step S9, the medical examination is ended, and the functional unit having the failure is repaired (step S17 in FIG. 7).

A Procedure for Generating/Displaying the Re-Interpretation Request Dialog

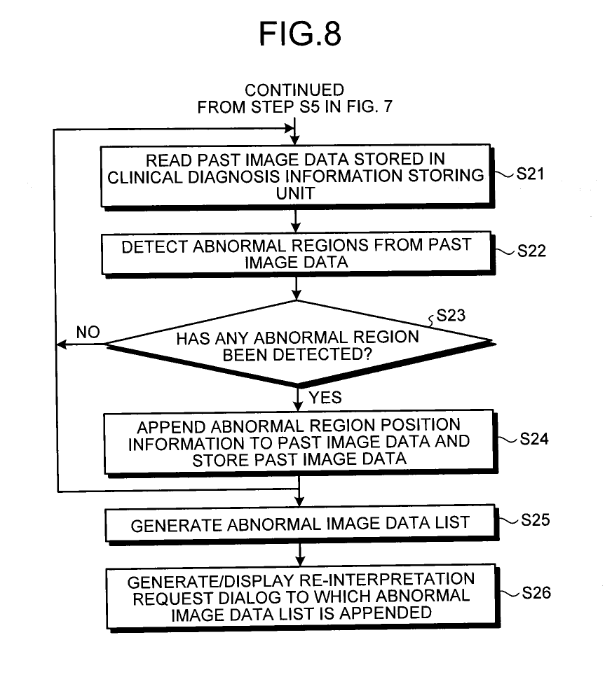

Next, a procedure for generating/displaying the re-interpretation request dialog according to the present embodiment will be explained, with reference to the flowchart in FIG. 8.

At step S7, when the one or more abnormal regions have been detected from the failure-diagnosis image data, and also, the detection result (the position information and the pixel values of the abnormal regions) has been stored, the data comparing unit included in the abnormal region detecting unit 11 reads the first piece of past image data that was acquired during a past medical examination using the medical image diagnosis apparatus 100 and is stored in the clinical diagnosis information storing unit 14 (step S21 in FIG. 8) and detects whether any abnormal region is present in the past image data or not, by comparing the pixel values of the past image data with the pixel values in the abnormal regions in the failure-diagnosis image data read from the abnormal region information storage unit 12 (step S22 in FIG. 8).

Further, if one or more abnormal regions have been detected from the first piece of past image data (step S23, YES in FIG. 7), the data appending unit included in the abnormal region detecting unit 11 appends the position information of the one or more abnormal regions to the past image data and stores the past image data back into the clinical diagnosis information storing unit 14 (step S24 in FIG. 8). On the contrary, if no abnormal region is detected at step S22, the first piece of past image data is stored in the clinical diagnosis information storing unit 14 without any modification applied thereto.

As a result of the procedure at steps S21 through S24 described above, when the one or more abnormal regions have been detected from the first piece of past image data, and also, the abnormal region position information has been appended to the first piece of past image data from which the abnormal regions were detected, the same process is performed on each of the other pieces of past image data stored in the clinical diagnosis information storing unit 14 (steps S21 through S24 in FIG. 8).

After that, when the above-mentioned process has been performed on each of all the pieces of past image data that were acquired by using the medical image diagnosis apparatus 100 and are stored in the clinical diagnosis information storing unit 14, the abnormal image list generating unit 15 generates a list (the abnormal image data list) showing one or more pieces of past image data each having one or more abnormal regions, on the basis of the identification information and the additional information thereof (step S25 in FIG. 8).

Further, the dialog generating unit included in the display data generating unit 16 generates a re-interpretation request dialog (see FIG. 6) that presents a message to request a re-interpretation of the pieces of past image data from which the one or more abnormal regions have been detected, as well as the abnormal image data list, and further causes the generated dialog to be displayed on the display unit 17 of the medical image diagnosis apparatus 100 or a terminal device used by a medical worker who is in charge of the interpretation of the past image data, the terminal device being connected via a network (not shown) or the like (step S26 in FIG. 8).

A Configuration of Another Apparatus

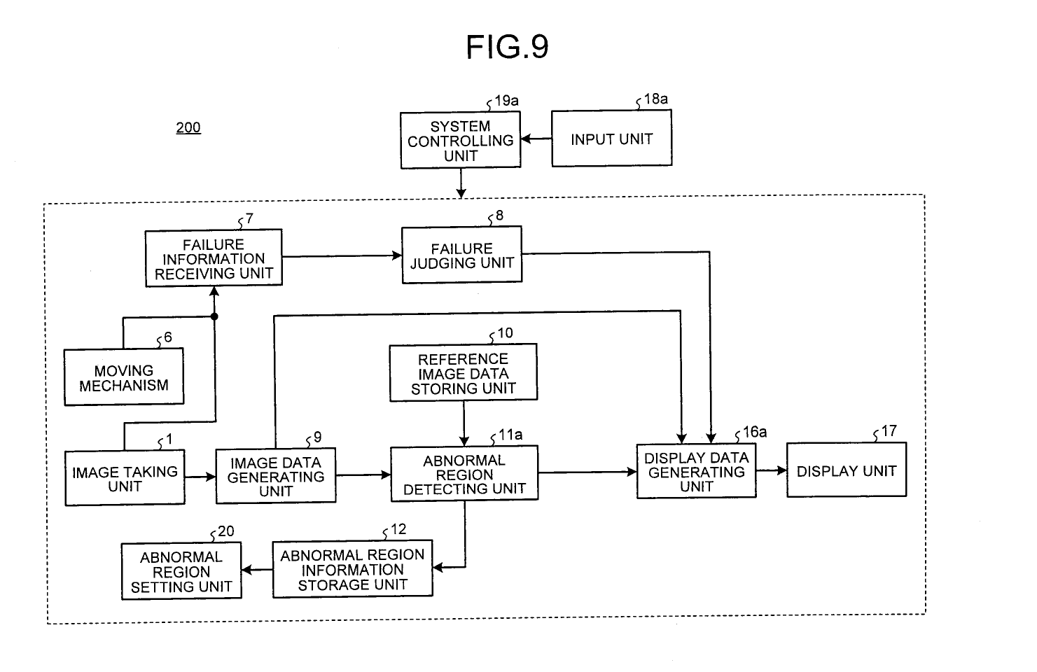

Next, a configuration and functions of a medical image diagnosis apparatus according to a modification example of the present embodiment will be explained, with reference to FIGS. 9 to 11. FIG. 9 is a block diagram of an overall configuration of the medical image diagnosis apparatus according to the present modification example. In FIG. 9, some of the functional units that have the same configurations and functions as those in the medical image diagnosis apparatus 100 shown in FIG. 1 will be referred to by using the same reference numerals, and the detailed explanation thereof will be omitted.

More specifically, a medical image diagnosis apparatus 200 according to the present modification example shown in FIG. 9 includes: the image taking unit 1 configured to generate projection data in the main image taking mode by performing an X-ray image taking process on an image taking region of the patient 150 and to further generate projection data in the failure diagnosis mode by performing a similar X-ray image taking process on an image taking region in which the patient 150 is not present; the holding unit (not shown) configured to hold the X-ray generating unit and the X-ray detecting unit (the image taking system) included in the image taking unit 1; the moving mechanism 6 configured to move the holding unit to which the image taking system is attached and the couchtop on which the patient 150 is placed in the predetermined directions; the failure information receiving unit 7 configured to receive the failure information supplied from the image taking unit 1 or the moving mechanism 6, as well as the failure speculation related information (i.e., the information about the warranty period, the use starting date, the medical examination dates, and the like) of the medical image diagnosis apparatus 200 that is supplied from a system controlling unit 19a (explained later) and is effective for speculating failures; the failure judging unit 8 configured to judge whether or not a failure detected or speculated is a failure that is harmful to the patient 150, the operator, or the like or a serious failure, on the basis of the failure information and the failure speculation related information; the image data generating unit 9 configured to generate clinical-diagnosis image data and failure-diagnosis image data by using the projection data supplied from the image taking unit 1 as a result of the X-ray image taking processes in the main image taking mode and the failure diagnosis mode; and the reference image data storing unit 10 configured to store therein reference image data acquired in advance while the patient 150 is not present, by using the medical image diagnosis apparatus 200 at a point in time when no failure had occurred therein.

The medical image diagnosis apparatus 200 further includes: an abnormal region detecting unit 11a configured to detect one or more abnormal regions from the failure-diagnosis image data, by comparing the failure-diagnosis image data generated by the image data generating unit 9 on the basis of the projection data in the failure diagnosis mode acquired during the X-ray image taking process performed on the image taking region in which the patient 150 is not present, with the reference image data read from the reference image data storing unit 10; the abnormal region information storage unit 12 configured to store therein position information of the one or more detected abnormal regions; an abnormal region setting unit 20 configured to set, for example, an optical spot corresponding to the position information of each of the abnormal regions read from the abnormal region information storage unit 12, so as to be positioned on or near the body surface of the patient 150 placed on the couchtop 160; a display data generating unit 16a configured to generate clinical-diagnosis display data by appending, as necessary, the patient information, the medical examination information, and the like to the clinical-diagnosis image data generated by the image data generating unit 9 in the main image taking mode and to further generate various types of dialogs on the basis of a result of the failure judgment by the failure judging unit 8, a result of the abnormal region detection by the abnormal region detecting unit 11a, and the like; the display unit 17 configured to display the clinical-diagnosis display data and the various types of dialogs generated by the display data generating unit 16a; an input unit 18a configured to input the patient information and the medical examination information, to set the various types of image taking conditions including the X-ray radiation condition, and to input the various types of instruction signals for, for example, instructing a failure diagnosis and instructing continuation/cancellation of medical examinations; and a system controlling unit 19a configured to control the functional units described above in an integrating manner.

The abnormal region detecting unit 11a includes a data comparing unit having the subtraction processing function and a data appending unit (not shown). The data comparing unit performs a subtraction process between the failure-diagnosis image data which the image data generating unit 9 generates on the basis of the projection data acquired during the X-ray image taking process in the failure diagnosis mode and the reference image data read from the reference image data storing unit 10. The data comparing unit thereby detects, for example, one or more abnormal regions caused by a failure in any of the functional units included in the medical image diagnosis apparatus 200 from the failure-diagnosis image data. Further, the position information, the pixel values, and the like of the detected abnormal regions are stored into the abnormal region information storage unit 12. Also, when one or more abnormal regions have been detected from the failure-diagnosis image data, the abnormal region detecting unit 11a supplies the instruction signal (the examination continuation judgment request instruction signal) to request that a judgment should be made as to whether the medical examination should be continued or not, to the dialog generating unit included in the display data generating unit 16a.

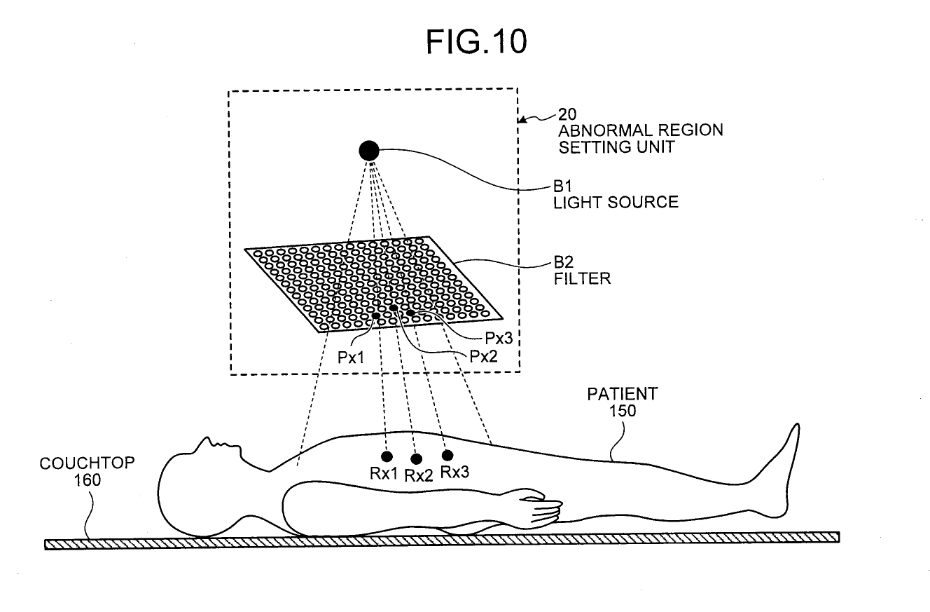

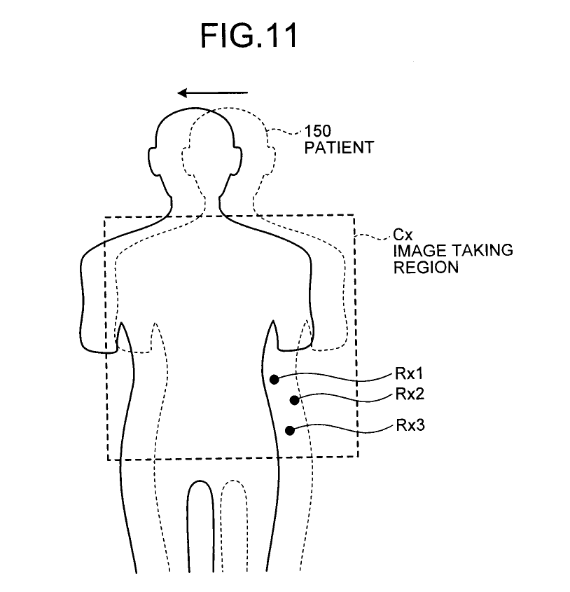

The abnormal region setting unit 20 includes: a light source B1 that is provided near the X-ray detecting unit 3, for example, and is configured to emit visible light in a direction toward the patient as shown in FIG. 10; and a filter B2 in which a plurality of holes capable of arbitrarily controlling transmission/blockage of the visible light are two-dimensionally arranged. In other words, the visible light that is emitted from the light source B1 and has passed through the holes of the filter B2 is radiated on or near the body surface of the patient 150 placed on the couchtop 160. In this situation, by allowing the visible light to pass only through holes Px1, Px2, and Px3 corresponding to abnormal regions Rx1, Rx2, and Rx3 in the failure-diagnosis image data, it is possible to set the abnormal regions Rx1, Rx2, and Rx3 so as to be positioned on or near the body surface of the patient 150. Accordingly, by moving the patient 150 in the direction of the arrow, as shown in FIG. 11, in such a manner that the abnormal regions Rx1, Rx2, and Rx3 set by the abnormal region setting unit 20 are positioned on the outside of a diagnosis target region of the patient 150, it is possible to acquire the clinical-diagnosis image data in an image taking region Cx, without being affected by the failure in the functional unit.

Returning to the description of FIG. 9, the display data generating unit 16a includes a data combining unit and a dialog generating unit (not shown). The data combining unit is configured to generate the clinical-diagnosis display data by appending, as necessary, the patient information, the medical examination information, the image taking condition, and/or the like to the clinical-diagnosis image data acquired during the X-ray image taking process in the main image taking mode. The dialog generating unit has a function of generating the various types of dialogs, on the basis of a result of the failure judgment by the failure judging unit 8, a result of the abnormal region detection by the abnormal region detecting unit 11a, and the like and is configured to generate the failure diagnosis request dialog to prompt a failure diagnosis for the purpose of evaluating the abnormal regions, on the basis of the failure diagnosis request instruction signal supplied from the failure judging unit 8. Further, the dialog generating unit is configured to generate the examination continuation judgment request dialog to request that a judgment should be made as to whether the medical examination should be continued or canceled, on the basis of the examination continuation judgment request instruction signal supplied from the abnormal region detecting unit 11a.

The input unit 18a is an interactive interface that includes one or more input devices such as a display panel, a keyboard, a trackball, a joystick, a mouse, and the like. The input unit 18a is configured to input the patient information and the medical examination information, to set the various types of image taking conditions including the X-ray radiation condition, to set the image data generation condition and the display data generation condition, and to input various types of instruction signals for, for example, instructing the failure diagnosis (to select the failure diagnosis mode) and instructing that the medical examination should be continued or canceled. For example, by using the input devices described above, the operator of the medical image diagnosis apparatus 200 selects the failure diagnosis mode in response to the failure diagnosis request dialog shown in FIG. 5A that was generated by the dialog generating unit included in the display data generating unit 16a and is displayed on the display unit 17, and also, instructs that the medical examination should be continued or canceled in response to the examination continuation judgment request dialog shown in FIG. 5B.

The system controlling unit 19a includes a CPU and an input information storage unit (not shown). The various types of information described above input/set by the input unit 18a are stored into the input information storage unit. Further, the input information storage unit has stored therein, in advance, the failure speculation related information such as the warranty period, the use starting date, the medical examination dates, and the like of the medical image diagnosis apparatus 200 that is effective for speculating failures. Further, the CPU controls the functional units described above included in the medical image diagnosis apparatus 200 in an integrating manner, on the basis of the stored information, so that the one or more abnormal regions are detected by comparing the failure-diagnosis image data with the reference image data, so that the abnormal regions are set with the patient 150 on the basis of the detection result, and so that the clinical-diagnosis image data is generated by performing the X-ray image taking process in the main image taking mode on the patient 150 whose position has been set on the basis of the abnormal regions set therewith.

A procedure for Generating/Displaying the Clinical-Diagnosis Image Data

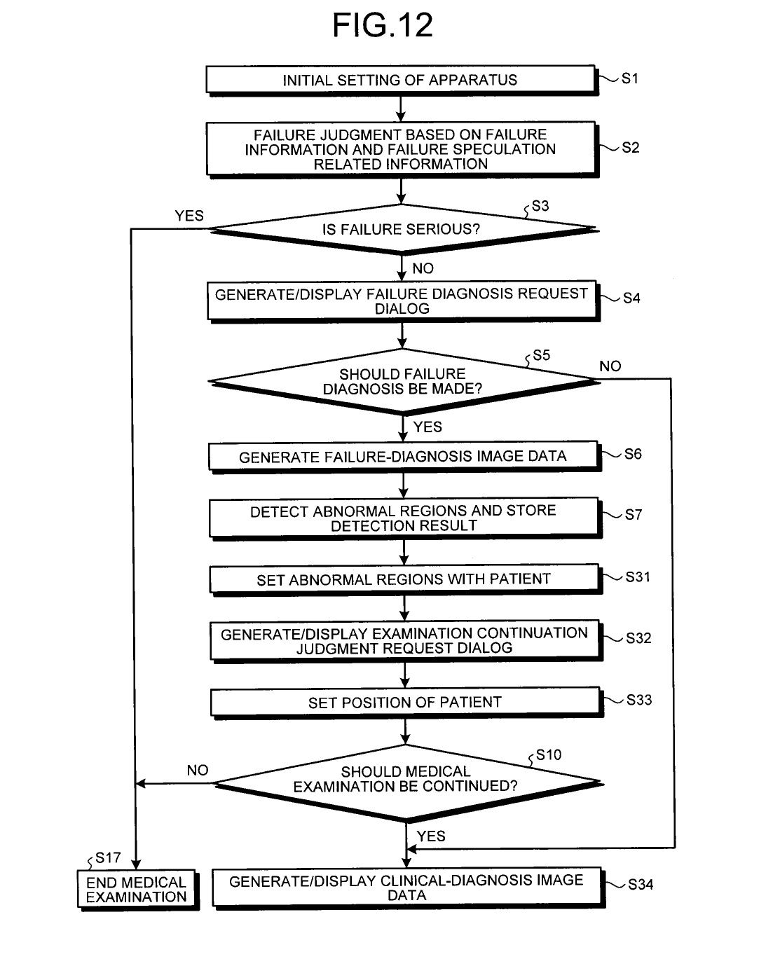

Next, a procedure for generating/displaying the clinical-diagnosis image data according to the modification example of the present embodiment will be explained, with reference to the flowchart in FIG. 12. In FIG. 12, some of the steps that are the same as those shown in FIG. 7 will be referred to by using the same reference numerals, and the detailed explanation thereof will be omitted.

More specifically, as a result of the procedure that is the same as that at steps S1 through S7 shown in FIG. 7, when the initial setting of the apparatus, the failure judgment based on the failure information and the failure speculation information, the generation/displaying of the failure diagnosis request dialog, the generation of the failure-diagnosis image data, the detection of the abnormal regions, and the storing of the detection result have been completed (steps S1 through S7 in FIG. 12), the abnormal region setting unit 20 causes the filter B2 to have such holes that allow transmission of visible light corresponding to the detection result (the position information of the abnormal regions) read from the abnormal region information storage unit 12.

After that, by causing the visible light that is emitted from the light source B1 and has passed through the holes of the filter B2 to radiate on the patient 150 placed on the couchtop 160, the abnormal region setting unit 20 sets abnormal regions corresponding to the abnormal regions detected from the failure-diagnosis image data so as to be positioned on or near the body surface of the patient 150 (step S31 in FIG. 12).

If a plurality of abnormal regions set with the patient 150 include one or more abnormal regions that are not caused by a failure in a functional unit, it is possible to eliminate those abnormal regions by an instruction signal input by the operator through the input unit 18a.

When the abnormal regions have been set with the patient 150, the abnormal region setting unit 20 supplies an examination continuation judgment request instruction signal to the dialog generating unit included in the display data generating unit 16a. Having received the instruction signal, the dialog generating unit generates an examination continuation judgment request dialog to request that a judgment should be made as to whether the medical examination should be continued or canceled and causes the generated dialog to be displayed on the display unit 17 (step S32 in FIG. 12).

The operator of the medical image diagnosis apparatus 200 sets the position and the posture of the patient 150 placed on the couchtop 160 in such a manner that the abnormal regions set on the body surface of the patient 150 are not included in a predetermined diagnosis region (step S33 in FIG. 12). After that, when having determined that it is possible to perform an medical examination on the patient 150 without being affected by the abnormal regions, the operator inputs an examination continuation instruction signal in response to the examination continuation judgment request dialog and selects the main image taking mode through the input unit 18a.

Subsequently, having received the instruction signal and the selection signal via the system controlling unit 19a, the image taking unit 1 generates projection data by performing an X-ray image taking process in the main image taking mode on the patient 150 placed on the couchtop 160. The image data generating unit 9 generates clinical-diagnosis image data on the basis of the obtained projection data and causes the generated image data to be displayed on the display unit 17 (step S34 in FIG. 12).

According to an aspect of the exemplary embodiments of the present disclosure described above, it is possible to prevent the clinical-diagnosis image data having one or more abnormal regions from being erroneously interpreted, by detecting, from the image data, the abnormal regions caused by the failure in the functional unit included in the medical image diagnosis apparatus by using the failure-diagnosis image data acquired during the image taking process in the failure diagnosis mode and by displaying the clinical-diagnosis image data acquired during the X-ray image taking process in the main image taking mode after appending thereto the warning data and the position information of the abnormal regions based on the detection result.

In particular, by displaying the position information of the abnormal regions caused by the failure in the functional unit so as to be superimposed on the clinical-diagnosis image data, it is possible to enable the viewer to carefully view image information of the clinical-diagnosis image data in the abnormal regions.

Accordingly, if the failure occurring in the medical image diagnosis apparatus is a failure that is not harmful to the patient or a minor failure, it is possible to continue to perform the medical examination. Thus, it is possible to avoid a significant delay in the medical examination that may be caused by substituting the apparatus with a replacement apparatus or repairing the defective location of the apparatus.

In addition, because the abnormal regions caused by the failure in the functional unit are detected by performing the subtraction process between the failure-diagnosis image data of the image taking region which was acquired by using the medical image diagnosis apparatus and in which the patient is not present and the reference image data of the image taking region which was acquired by using the medical image diagnosis apparatus at the point in time when no failure had occurred therein, it is possible to accurately detect the position information, the shape, and the like of the abnormal regions.

Moreover, the execution of the failure diagnosis, the continuation/cancellation of the medical examination, and the appending of the abnormal region position information to the clinical-diagnosis image data are performed on the basis of the instruction signals input by the operator in response to the various types of dialogs displayed on the display unit. Thus, the operator is able to input precise instructions to the medical image diagnosis apparatus quickly and easily. It is therefore possible to improve the efficiency in making diagnoses.

Further, according to an aspect of the exemplary embodiments described above, it is possible to detect whether any abnormal region is present in the past image data or not, by comparing the pixel values of the past image data acquired during the past medical examination by using the medical image diagnosis apparatus with the pixel values in the abnormal regions in the failure-diagnosis image data. Thus, it is possible to provide the medical worker who is in charge of interpreting the past image data with the information to prompt a re-interpretation of the past image data that has one or more abnormal regions. It is therefore possible to easily recognize or correct errors in the interpretations of the past image data having the one or more abnormal regions. In addition, by appending the position information of the abnormal regions detected by comparing the two pieces of image data with each other to the past image data, it is possible to enable the medical worker to efficiently re-interpret the past image data.

Furthermore, according to an aspect of the modification example described above, because region information corresponding to the abnormal regions detected by using the failure-diagnosis image data is set so as to be positioned on or near the body surface of the patient on whom the X-ray image taking process in the main image taking mode is to be performed, it is possible to obtain the clinical-diagnosis image data which is not affected by the failure in the functional unit and has high quality, by moving the patient in such a manner that the abnormal regions indicated on the body surface are positioned on the outside of the diagnosis target region.

The exemplary embodiments and the modification examples thereof of the present disclosure have thus been explained. It should be noted, however, that the present disclosure is not limited to the exemplary embodiments and the modification examples thereof described above. It is possible to embody the present disclosure by applying a further modification thereto. For example, in the exemplary embodiments and the modification examples thereof described above, the medical image diagnosis apparatuses 100 (or 200) configured to generate the clinical-diagnosis image data and the failure-diagnosis image data on the basis of the projection data acquired during the X-ray image taking process performed on the patient. However, the present disclosure is also applicable to other medical image diagnosis apparatuses configured to generate the above-mentioned image data by implementing other image taking methods such as an X-ray CT image taking method, an MRI image taking method, or the like.

Further, the examples are explained in which the failure-diagnosis image data and the reference image data are acquired by performing the X-ray image taking processes on the image taking region in which the patient 150 is not present; however, it is also acceptable to acquire the above-mentioned image data by performing an X-ray image taking process on a reference phantom, a model of a human body, or the like.

Further, the functional units from which a failure can be detected are not limited to the image taking unit 1 and the moving mechanism 6. Furthermore, it is also acceptable to further provide a display data printing unit and/or a display data storage unit configured to print and/or store therein the clinical-diagnosis display data (the first clinical-diagnosis display data) generated by appending the warning data and the position information of the abnormal regions to the clinical-diagnosis image data and the clinical-diagnosis display data (the second clinical-diagnosis display data) generated by appending the warning data to the clinical-diagnosis image data. Moreover, the failure speculation related information may be input during the initial setting at step S1.

Further, in the exemplary embodiments described above, the example is explained in which the abnormal regions are detected from the past image data stored in the clinical diagnosis information storing unit 14 included in the medical image diagnosis apparatus 100. It is, however, also acceptable to detect, in a similar procedure, the abnormal regions from the past image data that was acquired by using the medical image diagnosis apparatus 100 and is stored in a data server or the like connected via a network or a storage medium. In that situation, information about a transfer of the past image data to the data server is stored in the clinical diagnosis information storing unit 14, so that the abnormal region detecting unit 11 detects the abnormal regions from the past image data read from the data server on the basis of the transfer information. Furthermore, the example is explained in which the warning data is generated at step S6 in FIG. 7 immediately after the abnormal regions are detected; however, it is also acceptable to generate the warning data before or after the clinical-diagnosis image data is generated at step S8.

Further, the example is explained in which the clinical-diagnosis image data is generated after the abnormal regions are detected from the failure-diagnosis image data; however, it is also acceptable to generate the clinical-diagnosis image data prior to the failure judgment made on the basis of the failure information and the like and/or the failure diagnosis made for the purpose of detecting the abnormal regions.

Further, the example is explained in which, when the abnormal regions have been detected from the failure-diagnosis image data by the abnormal region detecting unit 11, the warning data generating unit 13 generates the warning data including the predetermined warning message; however, it is also acceptable to configure the warning data generating unit 13 so as to generate warning data that includes, instead of the warning message, a warning marker or the like that has a predetermined shape or a predetermined color tone.

In the modification example described above, the example is explained in which the abnormal regions caused by the failure in the functional unit are set so as to be positioned on or near the body surface of the patient 150 placed on the couchtop 160, by using the abnormal region setting unit 20 that includes the light source B1 configured to emit the visible light and the filter B2 in which the plurality of holes capable of arbitrarily controlling transmission/blockage of the visible light are two-dimensionally arranged; however, the present disclosure is not limited to this example. For example, another arrangement is acceptable in which the positions of the abnormal regions with respect to the patient 150 are displayed by obtaining taken image data by providing the X-ray tube 21 with an optical image taking unit such as a camera configured to take an image of the patient 150 and causing the display unit 17 to display a superimposed image obtained by superimposing the position information of the abnormal regions based on the detection result by the abnormal region detecting unit 11a onto the obtained taken image data.

It is possible to realize the functional units included in the medical image diagnosis apparatus 100 according to the present embodiment or the medical image diagnosis apparatus 200 according to the modification example, by employing a computer configured with, for example, a CPU, a Random Access Memory (RAM), a magnetic storage device, an input device, a display device, and the like as hardware. For example, it is possible to realize the various types of functions of the system controlling unit 19 controlling the functional units of the medical image diagnosis apparatus 100 or the system the system controlling unit 19a controlling the functional units of the medical image diagnosis apparatus 200, by causing the processor (e.g., the CPU) installed in the computer to execute a predetermined controlling program. In that situation, the controlling program may be installed in the computer in advance. Alternatively, the controlling program may be stored into a computer-readable storage medium or may be distributed via a network so as to be installed into the computer.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image diagnosis apparatus comprising:
an image data generating unit configured to generate clinical-diagnosis image data on a basis of taken image data obtained in a main image taking mode and to generate failure-diagnosis image data on a basis of taken image data obtained in a failure diagnosis mode;
an abnormal region detecting unit configured to detect, from the failure-diagnosis image data, an abnormal region caused by a failure in the apparatus, by comparing the failure-diagnosis image data with reference image data acquired in advance;
a warning data generating unit configured to generate warning data on a basis of a result of the abnormal region detection; and
a display unit configured to display the clinical-diagnosis image data to which the warning data is appended.

2. The medical image diagnosis apparatus according to claim 1, wherein
the image data generating unit generates the clinical-diagnosis image data on the basis of the taken image data obtained during an image taking process in the main image taking mode that is performed on a predetermined image taking region set with a patient, and
the image data generating unit generates the failure-diagnosis image data on the basis of the taken image data obtained during an image taking process in the failure diagnosis mode that is performed on an image taking region in which the patient is not present.

3. The medical image diagnosis apparatus according to claim 1, wherein the display unit displays the clinical-diagnosis image data to which position information of the abnormal region detected by the abnormal region detecting unit is further appended.

4. The medical image diagnosis apparatus according to claim 1, further comprising:
a failure judging unit configured to judge a degree of seriousness of the failure on a basis of either failure information or failure speculation related information of the medical image diagnosis apparatus; and
a selecting unit configured to select the failure diagnosis mode on a basis of a result of the judgment by the failure judging unit.

5. The medical image diagnosis apparatus according to claim 1, further comprising:
a clinical diagnosis information storing unit configured to store therein past image data acquired by the medical image diagnosis apparatus, wherein
the abnormal region detecting unit detects an abnormal region from the past image data, by comparing pixel values of the past image data with pixel values in the abnormal region in the failure-diagnosis image data.

6. The medical image diagnosis apparatus according to claim 1, wherein the abnormal region detecting unit detects an abnormal region from past image data that was acquired by the medical image diagnosis apparatus and was stored in advance into a data server connected via a network, by comparing pixel values of the past image data with pixel values in the abnormal region in the failure-diagnosis image data.

7. The medical image diagnosis apparatus according to claim 5, wherein the abnormal region detecting unit appends position information of the detected abnormal region to the past image data and stores the past image again.

8. The medical image diagnosis apparatus according to claim 6, wherein the abnormal region detecting unit appends position information of the detected abnormal region to the past image data and stores the past image again.

9. The medical image diagnosis apparatus according to claim 5, further comprising:
an abnormal image list generating unit configured to generate an abnormal image data list showing one or more pieces of past image data from which one or more abnormal regions have been detected by the abnormal region detecting unit; and
a dialog generating unit configured to generate a dialog presenting a message to prompt a re-interpretation of the one or more pieces of past image data from which the one or more abnormal regions have been detected and presenting the abnormal image data list.

10. The medical image diagnosis apparatus according to claim 6, further comprising:
an abnormal image list generating unit configured to generate an abnormal image data list showing one or more pieces of past image data from which one or more abnormal regions have been detected by the abnormal region detecting unit; and
a dialog generating unit configured to generate a dialog presenting a message to prompt a re-interpretation of the one or more pieces of past image data from which the one or more abnormal regions have been detected and presenting the abnormal image data list.

11. A medical image diagnosis apparatus comprising:
an image data generating unit configured to generate clinical-diagnosis image data on a basis of taken image data obtained in a main image taking mode and to generate failure-diagnosis image data on a basis of taken image data obtained in a failure diagnosis mode;
an abnormal region detecting unit configured to detect, from the failure-diagnosis image data, an abnormal region caused by a failure in the apparatus, by comparing the failure-diagnosis image data with reference image data acquired in advance; and
an abnormal region setting unit configured to set, with a patient, a region corresponding to the abnormal region detected from the failure-diagnosis image data.

12. The medical image diagnosis apparatus according to claim 11, wherein the abnormal region setting unit sets an optical spot in the region that corresponds to the abnormal region detected from the failure-diagnosis image data and is positioned on or near a body surface of the patient.

13. The medical image diagnosis apparatus according to claim 11, wherein the abnormal region setting unit sets the region corresponding to the abnormal region detected from the failure-diagnosis image data, with optical taken image data acquired of the patient.

14. A non-transitory computer-readable medium comprising a plurality of computer-executable instructions that cause a computer to operate a medical image diagnosis apparatus so as to perform the following, the medical image diagnosis apparatus being configured to acquire image data by performing a predetermined image taking process on a patient:
generating clinical-diagnosis image data on a basis of taken image data obtained in a main image taking mode and generating failure-diagnosis image data on a basis of taken image data obtained in a failure diagnosis mode;
detecting, from the failure-diagnosis image data, an abnormal region caused by a failure in the apparatus, by comparing the failure-diagnosis image data with reference image data acquired in advance;
generating warning data on a basis of a result of the abnormal region detection; and
displaying the clinical-diagnosis image data to which the warning data is appended.

15. A non-transitory computer-readable medium comprising a plurality of computer-executable instructions that cause a computer to operate a medical image diagnosis apparatus so as to perform the following, the medical image diagnosis apparatus being configured to acquire image data by performing a predetermined image taking process on a patient:
generating clinical-diagnosis image data on a basis of taken image data obtained in a main image taking mode and generating failure-diagnosis image data on a basis of taken image data obtained in a failure diagnosis mode;
detecting, from the failure-diagnosis image data, an abnormal region caused by a failure in the apparatus, by comparing the failure-diagnosis image data with reference image data acquired in advance; and
setting, with a patient, a region corresponding to the abnormal region detected from the failure-diagnosis image data.
